(19) United States
(12) Patent Application Publication
(10) Pub. No.: US 2018/0256449 A1
(43) Pub. Date: Sep. 13, 2018

(54) Device for Improving Treatment Efficacy and Patient Experience During Intravenous Infusions

(71) Applicant: The Happy Hanger, LLC, Greenville, NC (US)

(72) Inventor: Melanie Frost Moll, Greenville, NC (US)

(73) Assignee: The Happy Hanger, LLC, Greenville, NC (US)

(21) Appl. No.: 15/914,115

(22) Filed: Mar. 7, 2018

Related U.S. Application Data
(60) Provisional application No. 62/469,735, filed on Mar. 10, 2017.

(51) Int. Cl. A61J 1/14; A61M 5/14; A61J 1/16

(52) U.S. Cl. CPC A61J 1/1462; A61M 2205/59; A61J 1/16; A61M 5/1415

(57) ABSTRACT

An intravenous (IV) bag cover including a body of material; an IV pole/rack attachment mechanism disposed in the body of material; an ornamental feature disposed on at least a portion of one side of the body of material; and wherein the IV bag cover is configured such that when attached to an IV pole/rack it covers substantially all of at least one side of an IV bag installed on the IV pole/rack.

### RELATED APPLICATIONS

This application claims priority to and incorporates herein by reference related U.S. Provisional Patent Application No. 62/469,735 filed on Mar. 10, 2017.

### TECHNICAL FIELD

The presently disclosed subject matter relates generally to intravenous infusion treatments, and more particularly to a device for improving the efficacy of intravenous infusion treatments as well as the patient's experience and quality of life during such treatments.

### BACKGROUND

Stress and anxiety are well known to affect the efficacy of medical treatments, and can negatively impact a patient's treatment experience, attitude, and quality of life. The opposite has also been found to be true, that is positive attitude and reduced stress and anxiety have been shown to increase treatment efficacy and promotes healing. While battling an illness, such as cancer, a patient needs all the positivity they can get, and anything that promotes a positive attitude, reduces stress/anxiety, and instills hope can have a significant positive impact on not only the efficacy of the treatments, but on the patient's experience and overall quality of life during such a health battle.

It is all well known that the use of positive imagery can help to improve and promote a positive attitude, and can actually decrease stress and anxiety. Many offices and schools choose to have calming colors or murals painted on the walls or sides of their buildings to help facilitate a calming, relaxing, and less stressful atmosphere. Positive imagery and visualization, such as viewing pleasing and/or inspiring images can facilitate one's imagination, e.g., allow them to daydream and/or to mentally wander to a better place. By allowing a patient to focus on something pleasing, daydream, mentally wander to a better place, or visualize themselves doing something they aspire to do or experience once they recover, can all promote healing by improving the patient's attitude and inspiring hope during difficult and often stressful and exhausting treatments. The use of visualization and positive imagery can provide a better environment for the patient and improve the patient's outlook of the situation, thereby improving the patient's attitude and reducing their stress and anxiety.

Many medicines and treatments are delivered to a patient via intravenous (IV) infusion, which can be extremely daunting and unsettling both for the patient and caregiver. In particular, cancer treatment for children is intense and exhaustive, often requiring months of inpatient treatment. Such treatment typically involves the administration of chemotherapy via an IV bag. Over the course of a child's cancer treatment the child may have to receive hundreds of IV infusions. Many of the chemotherapy medicines are light sensitive and are therefore either sheathed in ominous looking light blocking bags or are of a fluorescent color (such as unpleasant shades of glow in the dark orange to harsh fluorescent yellows), which gives the IV bags a disturbing and unpleasant look. Just the sight of an IV pole with one or more IV bags dripping these unpleasant looking medicines into one's body can be terribly disturbing and invoke much stress and anxiety. In fact, it is not uncommon for pediatric cancer patients to become nauseous and vomit at the mere sight of the IV bag when it is brought into the room. This “anticipatory nausea” represents a well-known clinical phenomenon in oncology and is notoriously difficult to treat.

Therefore, there is a need for a device to improve the visual appearance of an IV bag, making it less daunting and/or to camouflage the medicines therein, thus, making the IV bag and its contents more aesthetically pleasing to a patient during IV infusions. Such a device could have the ability to improve the efficacy of IV delivered oncolytic agents by improving tolerance thereby facilitating dose density and intensity. Such a device could further improve the quality of life for both patients and caregivers during this challenging time.

### SUMMARY

In one embodiment, an intravenous (IV) bag cover is provided. The IV bag may include a body of material; an IV pole/rack attachment mechanism disposed in the body of material; an ornamental feature disposed on at least a portion of one side of the body of material; and wherein the IV bag cover is configured such that when attached to an IV pole/rack it covers substantially all of at least one side of an IV bag installed on the IV pole/rack. The body of material may be substantially opaque. The body of material may include a polypropylene material. The ornamental feature may include any one or more of a design, image, artwork, pattern, photograph, scene, sports logo, quote, and/or saying. The body of material may be the same size or slightly larger than the at least one side of the IV bag. The body of material may be in the range of about 4-9 inches wide by about 6-13 inches in length. The body of material may be substantially flat. The body of material may be configured to lay substantially flat immediately adjacent to a front surface of at least one side of the IV bag. The attachment mechanism may be disposed in an upper central portion of the body of material. The attachment mechanism may include a hole formed in the body of material. The hole maybe formed in an upper central portion of the body of material. The body of material may be in the range of about 0.009 mm to about 0.011 mm thick.

Certain aspects of the subject matter of the invention having been stated hereinabove, which are addressed in whole or in part by the disclosed subject matter, other aspects will become evident as the description proceeds when taken in connection with the accompanying Examples and Drawings as best described herein below.

### BRIEF DESCRIPTION OF THE DRAWINGS

Having thus described the subject matter of the invention in general terms, reference will now be made to the accompanying Drawings, which are not necessarily drawn to scale, and wherein:

FIG. 1 is an image of typical intravenous (IV) bags without a cover;

FIGS. 2 through 5 are images of IV bags with example aesthetically enhanced IV bag covers according to various embodiments of the invention; and

FIG. 6 is an image of a side by side comparison of typical IV bags without covers and the same IV bags with aesthetically enhanced covers according to various embodiments of the invention.

### DETAILED DESCRIPTION

The subject matter of the invention now will be described more fully hereinafter with reference to the accompanying Drawings, in which some, but not all embodiments of the disclosed subject matter are shown. Like numbers refer to like elements throughout. The disclosed subject matter may be embodied in many different forms and should not be construed as limited to the embodiments set forth herein; rather, these embodiments are provided so that this disclosure will satisfy applicable legal requirements. Indeed, many modifications and other embodiments of the disclosed subject matter set forth herein will come to mind to one skilled in the art to which the disclosed subject matter pertains having the benefit of the teachings presented in the foregoing descriptions and the associated Drawings. Therefore, it is to be understood that the disclosed subject matter is not to be limited to the specific embodiments disclosed and that modifications and other embodiments are intended to be included within the scope of the appended claims.

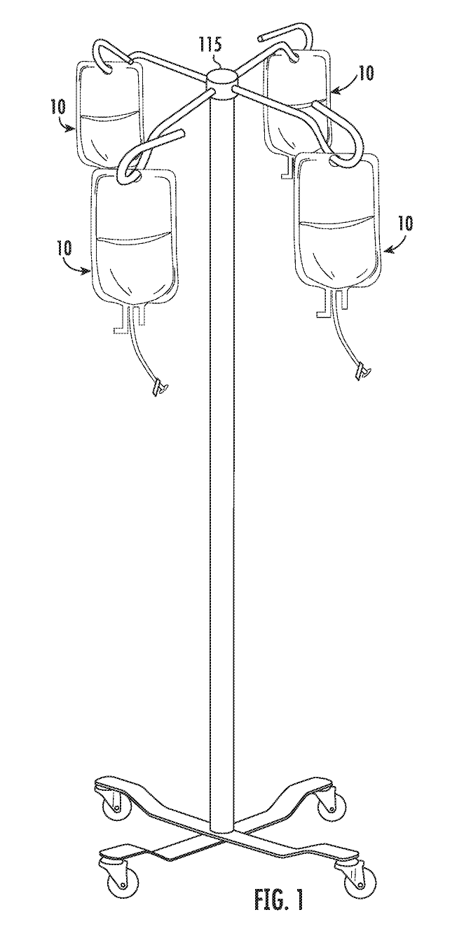

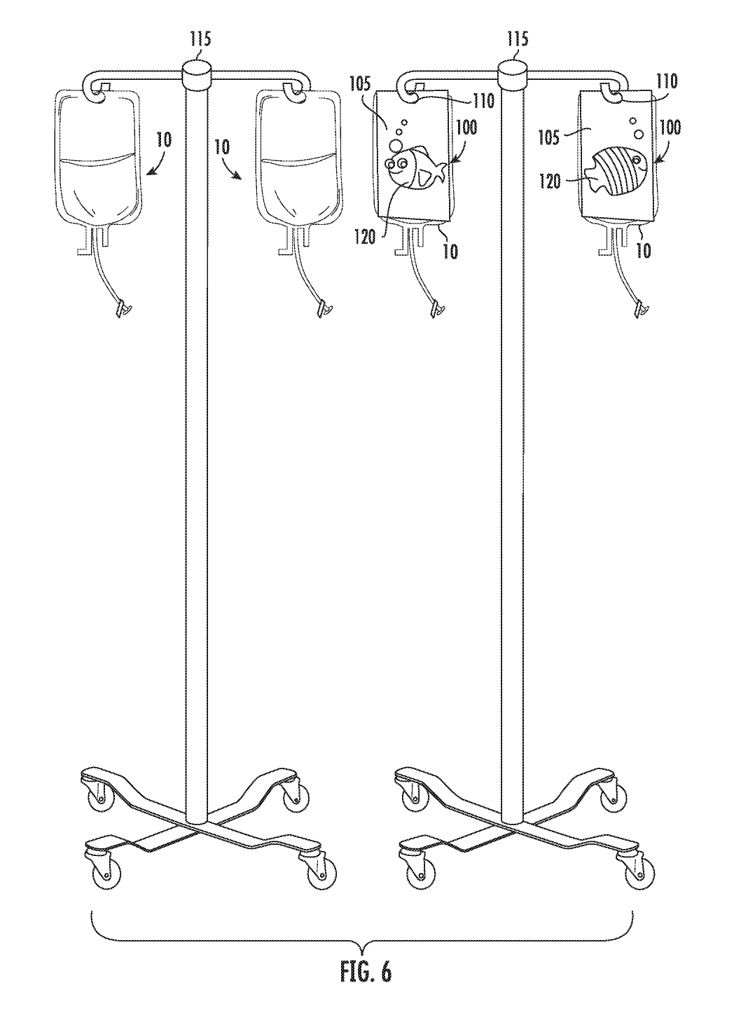

In one embodiment, a device for improving the efficacy of intravenous infusion treatments as well as a patient's experience and quality of life during such treatments is provided. Wherein the device may include an aesthetically enhanced IV bag cover that provides a much more aesthetically pleasing and therefore enhanced environment for a patient than a typically used uncovered IV bag, such as IV bags 10 shown in FIGS. 1 and 6; and especially as compared to uncovered IV bags that are used for delivery of light sensitive drug treatments, such as chemotherapy, which are delivered in dark or reflective ominous looking light blocking bags or the medication is of a fluorescent color (such as unpleasant shades of glow in the dark orange to harsh fluorescent yellows).

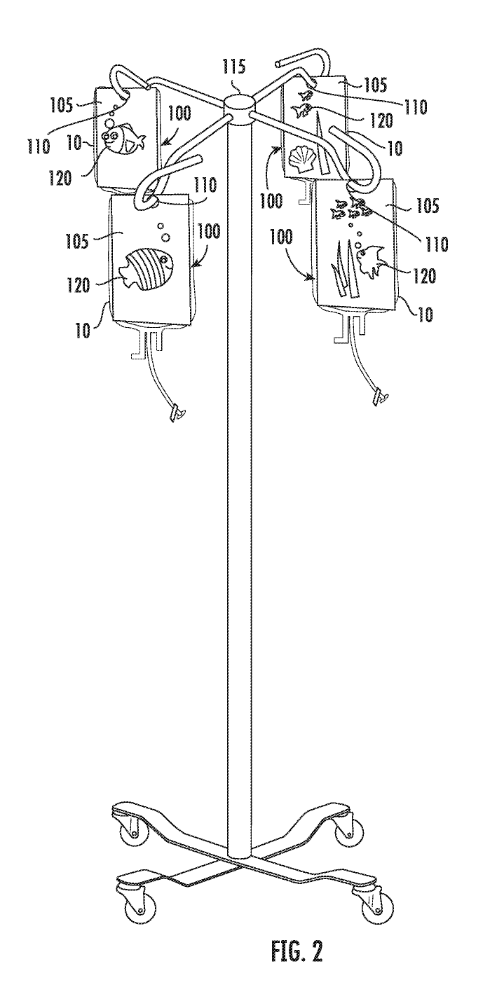

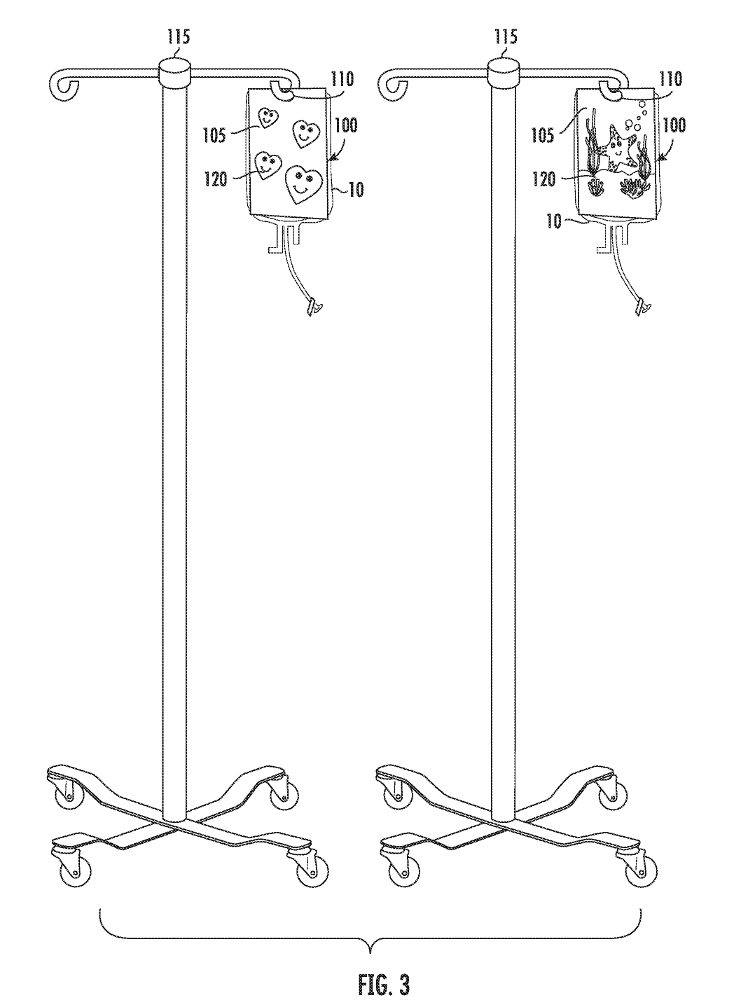

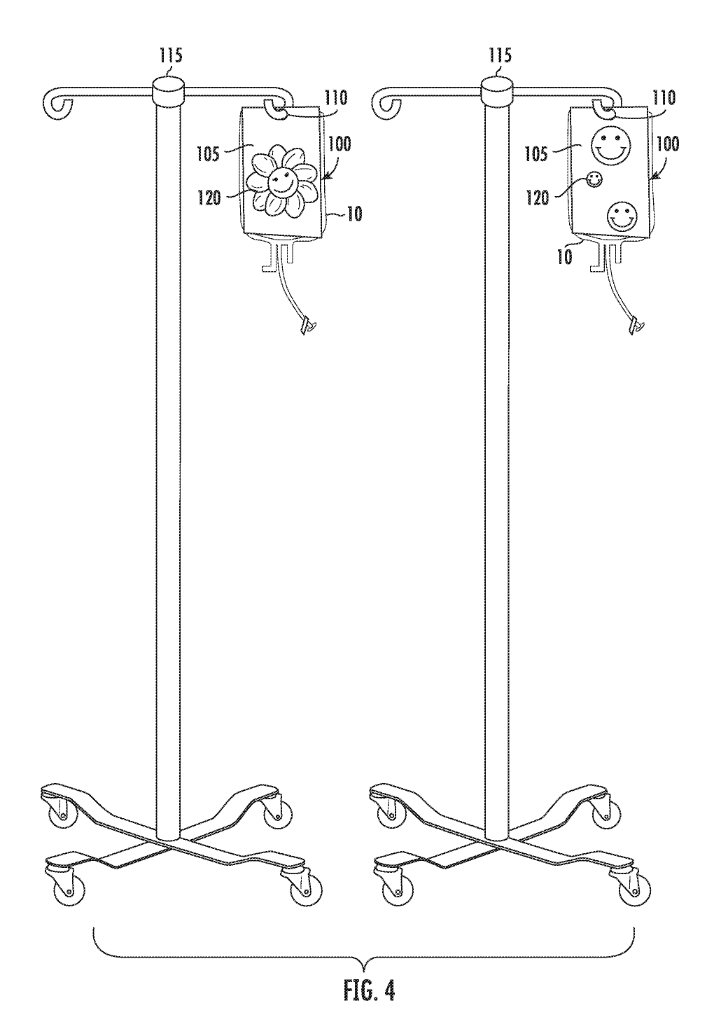

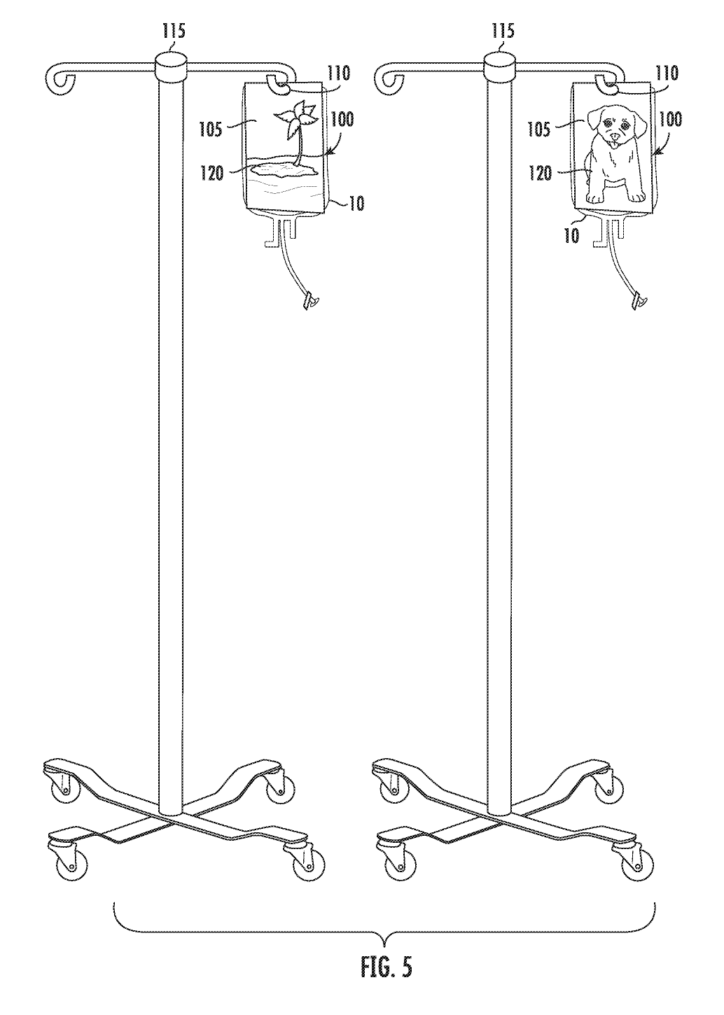

With reference now to FIGS. 2-5, in one embodiment the aesthetically enhanced IV bag cover 100 may include a body of material 105. The body of material 105 may be of any shape provided that it covers all, or substantially all, of at least one side of an IV bag 10, e.g., the patient facing side of the IV bag 10. As non-limiting examples only, aesthetically enhanced IV bag cover 100 may be generally square or rectangular shaped, or alternatively could even be in the shape of an animal or other object or design.

Aesthetically enhanced IV bag cover 100 may be reusable or disposable. Aesthetically enhanced IV bag cover 100 may be made of porous or nonporous material, such as plastic, fabric, paper, vinyl, metal, or any other suitable material. In one example, aesthetically enhanced IV bag cover 100 may be made of a polypropylene material. In another example, aesthetically enhanced IV bag cover 100 may be made of a material that meets specific OSHA standards and guidelines. In yet another example, aesthetically enhanced IV bag cover 100 may be made of a medical grade material or any other material suitable for use in a medical treatment facility. In still yet another example, aesthetically enhanced IV bag cover 100 may be made of the same material as that of a typical IV bag 10. In still yet another example, aesthetically enhanced IV bag cover 100 may be made of a non-DEHP latex free material. In still yet another example, aesthetically enhanced IV bag cover 100 may be made of synthetic non-woven material, such as banner material, for example, TexBanner material.

Aesthetically enhanced IV bag cover 100 may include an attachment mechanism 110 for attaching the aesthetically enhanced IV bag cover 100 to an IV pole/rack 115. Attachment mechanism 110 is preferably positioned in an upper portion of the aesthetically enhanced IV bag cover 100 and configured in a manner that when attached to the IV pole/rack 115 it hangs from the IV pole/rack 115 such that it covers all, or substantially all, of at least the patient facing portion of an IV bag 10 that is also hanging from the IV pole/rack 115. For example, attachment mechanism 110 may be a hole, hook, slit, clip, loop, or any other suitable mechanism, which is sized to easily engage with a standard IV pole/rack 115.

Aesthetically enhanced IV bag covers 100, are preferably opaque or substantially opaque, and as shown in FIGS. 2-5, may include any number of aesthetic enhancements, such as decorative images and/or designs 120, e.g., patterns, scenes, artwork, photos, etc., on at least the patient facing side. As non-limiting examples, decorative images and/or designs 120 may include depictions of tropical fish, sea life, happy hearts, brightly colored flowers, smiley faces, exotic locations, quotes or sayings, kid friendly characters, etc. The number of different images and/or decorative designs 120 is virtually unlimited, and may potentially include any kind of design, artwork, pattern, photograph, sports logos, quotes/sayings, and/or combinations thereof. Decorative images and/or designs 120 may include any number of themes, and may even be customize to a patient's preference. For example, aesthetically enhanced IV bag covers 100 may be customized to include pictures of loved ones, pets, and/or the like. Aesthetically enhanced IV bag covers 100 may also be used to provide hospital and/or patient identification and/or institutional advertising.

Aesthetically enhanced IV bag covers 100, may be sized and/or configure to cover almost any size IV bag 10, for example, but not limited to IV bags 10 in the range of about 50 ml to about 2,000 ml. In one example, aesthetically enhanced IV bag cover 100 may be in the range of about 4-9 inches wide by about 6-13 inches in length, and may have a thickness in the range of about 0.009 mm to about 0.011 mm.

Aesthetically enhanced IV bag covers 100 would preferably be substantially the same size as, or slightly larger than, the IV bag 10 it is covering, such that it does not unnecessarily interfere or get in the way of any other equipment or personnel. Additionally, multiple aesthetically enhanced IV bag covers 100 may be used with multiple IV bags 10 hanging on an IV pole or rack 115. Alternatively, a single large aesthetically enhanced IV bag cover 100 may be used to cover multiple IV bags 10 hanging on an IV pole or rack 115.

As can be seen in FIG. 6, which shows a side by side comparison of typical IV bags 10 without covers and IV bags 10 with aesthetically enhanced IV bag covers 100 attached thereto, aesthetically enhanced IV bag covers 100 help to provide a considerably improved and more pleasant experience for a patient by “camouflaging” the IV drip bag 10 with a decorative image and/or design 120, thus making it aesthetically more pleasant during an IV infusion treatment.

In use, the aesthetically enhanced IV bag covers 100 hang directly in front of the IV bag 10, without impeding the function or operation of the IV bag or the contents therein, while enhancing the appearance and overall look of the typical IV bag 10 delivery system to make it more aesthetically pleasing and improving the overall patient experience and ultimately improving the efficacy of the infusion treatment and healing.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A device for improving the efficacy of intravenous infusion treatments, comprising:
a. a body of material;
b. an IV pole/rack attachment mechanism disposed in the body of material;
c. an ornamental feature disposed on at least a portion of one side of the body of material; and
wherein the IV bag cover is configured such that when attached to an IV pole/rack it covers substantially all of at least one side of an IV bag installed on the IV pole/rack.

2. The IV bag cover of claim 1 wherein the body of material is substantially opaque.

3. The IV bag cover of claim 1 wherein the body of material comprises a polypropylene material.

4. The IV bag cover of claim 1 wherein the ornamental feature comprises any one or more of a design, image, artwork, pattern, photograph, scene, sports logo, quote, and saying.

5. The IV bag cover of claim 1 wherein the body of material is the same size or slightly larger than the at least one side of the IV bag.

6. The IV bag cover of claim 1 wherein the body of material is in the range of about 4-9 inches wide by about 6-13 inches in length.

7. The IV bag cover of claim 1 wherein the body of material is substantially flat.

8. The IV bag cover of claim 1 wherein the body of material is configured to lay substantially flat immediately adjacent to a front surface of at least one side of the IV bag.

9. The IV bag cover of claim 1 wherein the attachment mechanism is disposed in an upper central portion of the body of material.

10. The IV bag cover of claim 1 wherein the attachment mechanism comprises a hole formed in the body of material.

11. The IV bag cover of claim 10 wherein the hole is formed in an upper central portion of the body of material.

12. The IV bag cover of claim 1 wherein the body of material is in the range of about 0.009 mm to about 0.011 mm thick.
